(19) United States
(12) Patent Application Publication
(10) Pub. No.: US 2008/0260859 A1
(43) Pub. Date: Oct. 23, 2008

(54) Composition Containing Omega-3 Fatty Acids and Omega-6 Fatty Acids

(76) Inventors: Gudrun Claus-Herz, Berlin (DE); Guenther Bellmann, Berlin (DE)

Correspondence Address: Bausch & Lomb Incorporated, One Bausch & Lomb Place, Rochester, NY, 14604-2701, US

(21) Appl. No.: 11/577,149

(22) PCT Filed: Oct. 11, 2005
(86) PCT No.: PCT/EP05/55168

§ 371 Date: Jul. 9, 2008

(30) Foreign Application Priority Data
Oct. 13, 2004 (DE) 20 2004 015 931.7

(51) Int. Cl. A61K 31/202; A61K 31/20; A61K 31/34; A61K 31/355; A61K 31/675; A61K 31/7028; A61K 33/30; A61P 27/02

(52) U.S. Cl. 424/641; 514/560; 514/474; 514/458; 514/89; 514/52

(57) ABSTRACT

A composition comprises at least an omega-3 fatty acid, at least an omega-6 fatty acid, and zinc. The composition is advantageously administered to a subject suffering from a condition of dry eye. In certain embodiment, the amount of omega-3 fatty acid is much higher than that of the omega-6 fatty acid.

### BACKGROUND

The present invention relates generally to a composition containing omega-3 fatty acids. In particular, the present invention relates to such a composition that can be used as a dietary supplement or balanced diet supplement. More particularly, the present invention relates to the use of such a composition for the treatment of dry eye syndrome.

From a statistical point of view, every fifth patient seeking out an ophthalmologist practice suffers from dry eyes. The number of people suffering from this condition is estimated in Germany alone to be about 2 million. It is generally known that, in modern life, the eyes are subject to high stress, e.g. by screen handling, watching TV for a long time, or wearing of contact lenses or due to dry air from heaters or air conditioners. This stress can result inter alia in burning, itching or watering of the eyes. The reason for this is a disorder of the tear film caused by a high evaporation or a low tear production. Hormonal changes during aging, due to intake of certain medicaments (for example antibiotics, antihypertensives, antihistamines, vasoconstrictors, contraceptives, diuretics or antidepressants) or due to internal diseases such as Sjögren syndrome, rheumatism, or diabetes can also promote a dry eye condition.

Dry eye, which often results from the dysfunction of the sensitive system of tear production and tear distribution, requires continued treatment. Disorders of the tear film can be seen in a number of pathologies. The most frequent symptoms of dry eye include sensation of dryness or a feeling of a presence of a foreign body in the eye, or a feeling of pressure on the eye lid.

Normal tear secretion and normal tear flow are of substantial importance for the function and wellbeing of the eye. The tear film on the cornea has numerous important functions. For example, it produces a smooth cornea surface which is important for both the optical property as well as the movement of the eyes and the eye lids, prevents an irritation of the cornea due to dehydration, supports the supply of nutrients to the cornea and their metabolism, and mechanically removes foreign matter from the eye by frequent flushing. The tear film consists of the inner mucus layer, the intermediate aqueous layer, and the outer lipid layer.

Compositions containing omega-3 fatty acids are known in the art. WO 2004/004599 A3 (Advanced Vision Research) discloses a method for treatment of a condition selected from the group consisting of dry eye, irritation of Meibomian glands, dysfunction of Meibomian glands, and dry mouth. The method comprises administration of a dietary supplement, which contains an omega-6 (or n-6) fatty acid containing oil and omega-3 (or n-3) rich oil, wherein the n-3 rich oil has a high concentration of eicosapentaenoic acid (EPA) and a high concentration of docosahexaenoic acid (DHA). However, the teaching disclosed in WO 2004/004599 does not show optimal treatment results for the dry eye condition.

Therefore, there is a continued need to provide improved compositions for the amelioration or treatment of the dry eye condition. It is also very desirable to provide such compositions that have improved properties with respect to at least lessening the effects of irritation mediators in the eye. In addition, it is also very desirable to provide such compositions comprising ingredients that are economically and/or easily obtainable.

### SUMMARY

In general, the present invention provides a composition comprising: (a) at least an omega-3 fatty acid; (b) at least an omega-6 fatty acid; and (c) at least a zinc compound; wherein the composition is suitable for the amelioration or treatment of the dry eye syndrome.

In one aspect, said at least a zinc compound comprises zinc sulphate.

In another aspect, said at least an omega-3 fatty acid comprises low concentrations of eicosapentaenoic acid and/or docosahexaenoic acid. The present applicants have surprisingly discovered that such a composition comprising a high level of omega-3 and the inclusion of a zinc compound provides unexpected results in the amelioration or treatment of the dry eye condition. Although the applicants do not wish to be bound by any particular theory, they believe that the addition of zinc compounds possibly enhances the action of omega-6 fatty acids.

Other features and advantages of the present invention will become apparent from the following detailed description and claims.

### DETAILED DESCRIPTION

In general, the present invention provides a composition comprising: (a) at least an omega-3 fatty acid; (b) at least an omega-6 fatty acid; and (c) at least a zinc compound; wherein the composition is suitable for the amelioration or treatment of the dry eye syndrome.

In one aspect, said zinc compound is a pharmaceutically acceptable zinc compound.

Although the applicants do not wish to be bound to any particular theory, they believe that the addition of zinc compounds has a major influence on the improved treatment of the dry eye syndrome when simultaneously used with omega-3 fatty acids and omega-6 fatty acids. For example, omega-3 and omega-6 fatty acids contained in the compositions of the present invention metabolize in the body inter alia to prostaglandins PGE1 and PGE3, which have anti-inflammatory effects. There has been growing evidence that the dry eye condition can have an aetiology in inflammation. The addition of zinc in the form of a zinc compound in the inventive compositions containing omega-3 fatty acids and omega-6 fatty acids promotes the conversion of these fatty acids into PGE1 and PGE3, leading to improved results in the treatment of the dry eye syndrome.

In one aspect of the present invention, the use of omega-3 fatty acids having a high concentration of eicosapentaenoic acid (EPA) and/or a high concentration of docosahexaenoic acid (DHA) in combination with at least one zinc compound appears to have a surprisingly good synergistic effect in the treatment of the dry eyes syndrome.

Furthermore, the use of the inventive composition comprising omega-3 and omega-6 fatty acids in combination with zinc compounds may cause an increased production of precursor compounds necessary for the formation of lipids of the tear film.

Unless otherwise indicated, the concentration of an ingredient as disclosed herein, for example, in weight percent, is based on the total weight of the composition without the shell that encloses the composition, when such composition is in a solid form.

A composition of the present invention comprises a concentration of total omega-3 fatty acids in a range from about 1 to about 85 percent by weight, calculated as triglycerides. Alternatively, such concentration of total omega-3 fatty acids is in the range of about 5 to about 75, or from about 15 to about 65, or from about 30 to about 50, or from about 45 to about 50 percent by weight, calculated as triglycerides.

In another aspect, a composition comprises a concentration of total omega-6 fatty acids in a range from about 0.1 to about 5 percent by weight of the total composition. Alternatively, such concentration of total omega-6 fatty acids is in the range of about 0.3 to about 2, or from about 0.5 to about 1.5, or from about 0.5 to about 1, or from about 0.75 to about 1 percent by weight of the total composition.

In still another aspect, the composition comprises total omega-3 fatty acids and total omega-6 fatty acids in any combination of concentration ranges disclosed above.

In one embodiment, the omega-3 fatty acids comprise eicosapentaenoic acid (EPA) in a range from about 25 to about 75 percent by weight of the total composition, calculated as triglycerides. Alternatively, the eicosapentaenoic acid is in a range from about 30 to about 70, or from about 40 to about 60, or from about 45 to about 55, or from about 47 to about 52, or from about 49 to about 51 percent by weight of the total composition, calculated as triglycerides.

In another embodiment, the omega-3 fatty acids comprise docosahexaenoic acid (DHA) in a range from about 3 to about 60 percent by weight of the total composition, calculated as triglycerides. Alternatively, DHA is in a range from about 10 to about 50, or from about 20 to about 40, or from about 30 to about 35, or from about 32 to about 34 percent by weight of the total composition, calculated as triglycerides.

In still another embodiment, a composition comprises EPA and DHA, each at a concentration in any respective range disclosed above.

In yet another embodiment, the omega-6 fatty acids comprise gamma-linolenic acid (GLA) in a concentration in the range from about 3 to about 50 percent by weight of the total omega-6 fatty acids included in the composition. Alternatively, gamma-linolenic acid comprises from about 5 to about 40, or from about 10 to about 30, or from about 15 to about 25, or from about 17 to about 19 percent by weight of the total omega-6 fatty acids included in the composition.

Suitable omega-3 fatty acids usable in a composition of the present invention may be selected from the group consisting of alpha-linolenic acid, stearidonic acid, eicosapentaenoic acid, docosapentaenoic acid, docosahexaenoic acid, and combinations thereof.

Suitable omega-6 fatty acids usable in a composition of the present invention may be selected from the group consisting of linoleic acid, gamma-linolenic acid, dihomo-gamma-linolenic acid, and combinations thereof.

In a further aspect, a composition of the present invention further comprises a zinc compound. In general, any pharmaceutically acceptable zinc compound can be used. For example, suitable are pharmaceutically acceptable inorganic and/or organic salts of zinc, such as zinc sulphate monohydrate, zinc sulphate septahydrate, zinc histidinate-dihydrate, zinc chloride, zinc asparatate, zinc gluconate, zinc orotate, zinc oxide, or a combination thereof may be used according to the invention. In one embodiment, the zinc compound is zinc sulphate.

Preferably, a zinc compound is added to the composition in an amount so as to give an amount of zinc in a range from about 0.01 to about 10 mg, or from about 1 to about 6 mg, or from about 2 to about 4 mg, or from about 3 to about 3.5 mg, or from about 3.3 to about 3.4 mg. This information is preferably based on a single dose of the composition. In other preferred embodiments, the composition contains an amount of zinc in the range from about 2.5 to about 5 mg based on a single dose of the composition.

In another aspect, a composition of the present invention comprises an amount of zinc at a concentration in a range from about 0.01 to about 2 percent by weight of the total composition. Alternatively, zinc is present at a concentration in a range from about 0.1 to about 1, or from about 0.3 to about 0.7, or from about 0.5 to about 0.6, or from about 0.55 to about 0.57 percent based on the total weight of the composition.

In another embodiment, the composition contains at least one vitamin. In a preferred embodiment of the present invention, the composition contains at least one vitamin selected from the group consisting of vitamin E, vitamin C, vitamin B, wherein B6, vitamin B12, and combinations thereof. Lipophilic vitamin E may inter alia act as antioxidant and thus may prevent the omega-3 and omega-6 fatty acids contained in the composition from oxidation. Both vitamin C as well as, especially, vitamin B6 can provide further improved or desirable effects of a composition of the invention.

Further advantages of the composition of the invention can be achieved by using vitamin B6 and/or vitamin B12 in combination with omega-3 and omega-6 fatty acids and zinc. This combination can advantageously have a positive effect on tear production. The applicants believe that the use of vitamin B6 and/or vitamin B12 in combination with omega-3 and omega-6 fatty acids and zinc can contribute to the maintenance of the natural tear film in the eye and to the improvement of the supply of natural moisture to the eye.

For a further improvement of the effect of the composition, an amount of vitamin B6 in a range from about 0.1 to about 1 mg has been found to be advantageous for a single dose of the composition. Alternatively, an amount of vitamin B6 is in a range from about 0.3 to about 0.8 mg, or from about 0.5 to about 0.75 mg, or from about 0.6 to about 0.7 mg, based on a single dose of the composition.

In still other embodiments, the composition further comprising vitamin B12 may be preferred. During research, the applicants have discovered that an amount of vitamin B12 greater than 10 μg for the single dose caused an unfavourable change in the pharmaceutical acceptability of the composition. Therefore, a composition of the present invention preferably comprises, based on a single dose, an amount of vitamin B12 in a range from about 0.01 to about 1 μg, or from about 0.1 to about 1 μg, or from about 0.1 to about 0.7 μg, or from about 0.25 to about 0.5 μg, or from about 0.3 to about 0.35 μg, or from about 0.32 to about 0.34 μg.

It has further been found that adding vitamin C to a composition can yield a further improvement. Thus, in a further embodiment of the invention, a composition further comprises an amount of vitamin C in a range from about 1 to about 50 mg, or from about 10 to about 40 mg, or from about 15 to about 30 mg, or from about 15 to about 25 mg, or from about 19 to about 21 mg.

In especially preferred embodiments, the composition comprises omega-3 fatty acids, gamma-linolenic acid, vitamin C, vitamin E, vitamin B6, Vitamin B12 and zinc.

In further preferred embodiments, the composition comprises fish oil, borage seed oil, vitamin C, vitamin E, vitamin B6, vitamin B12 and zinc.

In a further preferred embodiment of the invention the composition of a single dosage comprises:

(a) at least an omega-3 acid in an amount from about 10 about 500 mg, preferably from about 100 to about 400 mg, more preferably from about 200 to about 300 mg, especially preferably from about 280 to about 290 mg, calculated as triglycerides;

(b) gamma-linolenic acid in an amount from about 1 to about 10 mg, preferably from about 2 to about 8 mg, more preferably from about 4 to about 6 mg, especially preferably from about 4.5 to about 5.5 mg;

(c) vitamin E in an amount from about 1 to about 5 mg, preferably from about 2 to about 4.5 mg, more preferably from about 3 to about 4 mg, especially preferably from about 3.1 to about 3.5 mg vitamin E;

(d) vitamin C in an amount from about 1 to about 50 mg, preferably from about 10 to about 40 mg, more preferably from about 15 to about 30 mg, especially preferably from about 19 to about 21 mg vitamin C;

(e) vitamin B₆ in an amount from about 0.1 to about 1 mg, preferably from about 0.3 to about 0.8 mg, more preferably from about 0.5 to about 0.75 mg, especially preferably from about 0.6 to about 0.7 mg;

(f) vitamin B₁₂ in an amount from about 0.01 to about 1 μg, preferably from about 0.15 to about 0.7 μg, more preferably from about 0.25 to about 0.5 μg, especially preferably from about 0.32 to about 0.34 μg; and

(g) zinc in an amount from about 0.1 to about 10 mg, preferably from about 1.5 to about 5 mg, more preferably from about 2 to about 4 mg, especially preferably from about 3.3 to about 3.4 mg.

In another embodiment, the composition can further comprises mixed tocopherol in an amount from about 0.01 to about 5 mg, preferably from about 0.1 to about 3 mg, more preferably from about 0.5 to about 2 mg, especially preferably from about 0.9 to about 1.1 mg.

In still another preferred embodiment, the composition comprises, based on a single dose, 285 mg omega-3 fatty acids calculated as triglycerides, 5 mg gamma-linolenic acid, 20 mg vitamin C, 3.5 mg vitamin E, 0.67 mg vitamin B6, 0.34 μg vitamin B12, and 3.3 mg zinc.

In still another preferred embodiment, the composition comprises, based on a single dose, 475 mg fish oil, 29.4 mg borage seed oil, 20 mg vitamin C, 3.5 mg vitamin E, 0.67 mg vitamin B₆, 0.34 μg vitamin B₁₂, and 3.33 mg zinc.

In still another preferred embodiment, the composition the composition comprises, based on a daily dose, 855 mg omega-3 fatty acids calculated as triglycerides, 15 mg gamma-linolenic acid, 60 mg vitamin C, 10 mg vitamin E, 2 mg vitamin B6, 1 μg vitamin B12, and 10 mg zinc.

In still another preferred embodiment, the composition comprises, based on a daily dose, 1425 mg fish oil, 88 mg borage seed oil, 60 vitamin C, 10 mg vitamin E, 2 mg vitamin B6, 1 μg vitamin B12, and 10 mg zinc.

A composition of the present invention provides the body with extra supply of material for the biosynthesis of PGE₁ and PGE₃. In one aspect, the supply of omega-3 and omega-6 fatty acids in combination with one or more zinc compounds, as well as vitamin C and one or more B-vitamins shows a significant improvement in the treatment or dietary treatment, respectively, of the dry eye syndrome.

Suitable fish oil employed in a composition of the present invention comprises oil from fish and/or sea animals.

The composition of the invention may preferably contain the omega-3 fatty acids in the form of triglycerides of fish oil. Most preferably, the omega-3 fatty acid is present as eicosapentaenoic acid and/or docosahexaenoic acid which preferably are obtainable from the following: rape oil, linseed and its oil, and/or fish oil. The omega-6 fatty acid may be gamma-linolenic acid, which preferably is obtainable from borage seed oil, evening primrose oil, and/or core oil of black currants. Furthermore, the composition of the invention may contain adjutants selected from the group comprising glycerol monostearate, lecithin, and/or gelatin.

In a preferred embodiment of the invention, the weight ratio of omega-3 fatty acids calculated as triglycerides and gamma-linolenic acid is from about 3:1 to about 200:1, preferably from about 20:1 to about 100:1, more preferably from about 50:1 to about 60:1, especially preferably from about 56:1 to about 58:1. It is believed that the omega-3 fatty acid eicosapentaenoic acid competes for the enzyme delta-5-desaturase against the dihomo-gamma-linolenic acid derived from the class of omega-6 fatty acids. Thus, the excess of eicosapentaenoic acid in the present compositions compared to those of the prior-art not only increases the synthesis of PGE₃, but also indirectly competes for the action of delta-5-desaturase, resulting in a reduced synthesis of the undesired arachidonic acid, and consequently, a reduced synthesis of the undesired PGE₂. Furthermore, excess EPA can be converted to the desirable PEG₃ in a cyclooxygenase pathway. The addition of a zinc compound and optionally vitamins here has a positive and synergistic effect which is not yet completely understood.

Thus, the components or the composition of the invention as a whole cause a positive influence on the relative ratios of inflammation mediators, i.e. the ratios of the desired anti-inflammatory and tear secretion improving PGE1 and PGE3 on the one hand and the undesired inflammation promoting PGE2 on the other hand.

Moreover, it is advantageous that the dosage forms of the compositions of the invention have no adverse side effects and are suitable for the dietary prophylaxis and/or treatment of diseases of the eye, preferably for the treatment of the dry eye syndrome. A positive effect can be seen especially when using a composition of the present invention as a supplementary balanced diet or as a dietary supplement for the treatment or amelioration of conditions resulting from deficiencies of these nutrients and for the prophylaxis of nutritional deficiencies that may cause the dry eye syndrome.

The composition containing omega-3 fatty acids may be solid, liquid and/or in the form of a gel; preferably the composition is embodied in a pharmaceutical form selected from the group consisting of tablets, coated tablets, dragees, capsules, powder, granulate, solutions, and effervescent tablets.

The materials are preferably used in a form adapted to the selected pharmaceutical form. The composition containing omega-3 fatty acids preferably includes vitamin E as (R, R, R)-alpha-tocopherol and/or as a mixed concentrate of tocopherol. The composition preferably includes vitamin C as calcium ascorbate. In especially preferred embodiments, the composition containing omega-3 fatty acids includes fish oil triglycerides, borage seed oil, (R, R, R)-alpha-tocopherol, mixed concentrate of tocopherol, calcium ascorbate, pyridoxine hydrochloride, zinc sulphate monohydrate and cyanocobalamine, glycerol monostearate, lecithin and/or gelatin.

Preferably, the omega-3-fatty acid containing composition is provided especially as dietary supplement in the form of a capsule, especially a gelatin capsule. The capsule comprises preferably a capsule shell having a weight of from about 50 to about 500 mg, preferably from about 100 to about 400 mg, more preferably from about 218 to about 256 mg, especially preferably from about 237 to about 238 mg. The capsule shell can have an adjuvant selected from the group consisting of glycerine, gelatine, dyes, e.g. red iron oxide, and combinations thereof. Further suitable dyes are preferably selected from group consisting of triarylmethane dyes, preferably brilliant blue; azo dyes, preferably allura red; and titanium dioxide.

Furthermore, in a preferred embodiment of the composition of the invention the content of the pharmaceutical form has a total weight of from about 1 to about 1500 mg, or from about 200 to about 1500 mg, or from about 200 to about 1000 mg, preferably from about 200 to about 800 mg, more preferably from about 400 to about 700 mg, especially preferably from about 596 to about 598 mg. In a further preferred embodiment, the content of the dosage form, e.g. the content of a capsule, has a total weight of from about 600 to about 605 mg especially preferably of 602 mg.

The daily dose of the composition of the invention may consist of one or several single doses. For example, the daily dose may consist of 2, 3, 4, or more single doses.

The subject matter of the present invention will further be illustrated with reference to examples 1 to 12 below.

### EXAMPLE 1

A single dose contains:

| | | |
|---|---|---|
| Fish oil | 475 | mg |
| Omega-3 fatty acids (calculated as triglycerides) | 285 | mg |
| Borage seed oil | 29.4 | mg |
| Gamma-linolenic acid | 5 | mg |
| (R,R,R)-alpha-tocopherol concentrate | 3.334 | mg |
| Ascorbic acid (as calcium ascorbate) | 20 | mg |
| Vitamin B6 (as pyridoxine hydrochloride) | 0.667 | mg |
| Zinc (as zinc sulphate monohydrate) | 3.34 | mg |
| Cyanocobalamine | 0.33 | μg |
| Glycerol monostearate, lecithin, gelatin | 597 | mg |

Further conventional adjuvants such as stabilizers and/or stability additives and/or production additives are contained in addition to the indicated ingredients.

### EXAMPLE 2

One capsule shell contains glycerol, gelatine, and red iron oxide in a total amount of 237.2 mg.

Further conventional adjuvants such as stabilizers and/or stability additives and/or additives for facilitating production are contained in addition to the indicated ingredients.

### EXAMPLE 3

A recommended daily dose of 3 capsules contains:

| | | |
|---|---|---|
| Fish oil | 1425 | mg |
| Omega-3 fatty acids (calculated as triglycerides) | 855 | mg |
| Borage seed oil | 88 | mg |
| Gamma-linolenic acid | 15 | mg |
| (R,R,R)-alpha-tocopherol concentrate | 10 | mg |
| Ascorbic acid (as calcium ascorbate) | 60 | mg |
| Vitamin B6 (as pyridoxinehydrochloride) | 2 | mg |
| Zinc (as zinc sulphate monohydrate) | 10 | mg |
| Cyanocobalamine | 1 | μg |
| Glycerol monostearate, lecithin, gelatin | 1791 | mg |

Further conventional adjuvants such as stabilizers and/or stability additives and/or production additives are included, if desired, in addition to the indicated ingredients.

### EXAMPLE 4

Three capsule shells of the recommended daily dose contain glycerol, gelatine, and red iron oxide in a total amount of 712 mg.

Further conventional adjuvants such as stabilizers and/or stability additives and/or production additives are included, if desired, in addition to the indicated ingredients.

### EXAMPLE 5

A single dose contains:

| | | |
|---|---|---|
| Fish oil | 475 | mg |
| Omega-3 fatty acids (calculated as triglycerides) | 285 | mg |
| Borage seed oil | 29.4 | mg |
| Gamma-linolenic acid | 5 | mg |
| (R,R,R)-alpha-tocopherol concentrate | 3.334 | mg |
| Ascorbic acid (as calcium ascorbate) | 20 | mg |
| Vitamin B6 (as pyridoxinehydrochloride) | 0.667 | mg |
| Zinc (as zinc sulphate monohydrate) | 3.34 | mg |
| Cyanocobalamine | 0.33 | μg |

wherein glycerol monostearate, lecithin, and gelatine are added in a total amount of 602 mg.

Further conventional adjuvants such as stabilizers and/or stability additives and/or production additives are included, if desired, in addition to the indicated ingredients.

### EXAMPLE 6

A capsule shell of 237.2 mg composed of glycerol, gelatine, red iron oxide. Further conventional adjuvants such as stabilizers and/or stability additives and/or production additives are included, if desired, in addition to the indicated amounts.

### EXAMPLE 7

A single dose contains:

| | | |
|---|---|---|
| Fish oil | 475 | mg |
| Omega-3 fatty acids (calculated as triglycerides) | 285 | mg |
| Borage seed oil | 29.4 | mg |
| Gamma-linolenic acid | 5 | mg |
| (R,R,R)-alpha-tocopherol concentrate | 3.334 | mg |
| Ascorbic acid (as calcium ascorbate) | 20 | mg |
| Vitamin B6 (as pyridoxinehydrochloride) | 0.667 | mg |
| Zinc (as zinc sulphate monohydrate) | 3.34 | mg |
| Cyanocobalamine | 0.33 | μg |

wherein glycerol monostearate, lecithin, and gelatine are added in a total amount of 602 mg.

Further conventional adjuvants such as stabilizers and/or stability additives and/or production additives are included, if desired, in addition to the indicated ingredients.

### EXAMPLE 8

A capsule shell of 237.87 mg composed of glycerol, gelatine, brilliant blue dye.

Further conventional adjuvants such as stabilizers and/or stability additives and/or production additives are included, if desired, in addition to the indicated ingredients.

### EXAMPLE 9

A recommended dose of 3 capsules contains:

| | | |
|---|---|---|
| Fish oil | 1425 | mg |
| Omega-3 fatty acids (calculated as triglycerides) | 855 | mg |
| Borage seed oil | 88 | mg |
| Gamma-linolenic acid | 15 | mg |
| (R,R,R)-alpha-tocopherol concentrate | 10 | mg |
| Ascorbic acid (as calcium ascorbate) | 60 | mg |
| Vitamin B6 (as pyridoxinehydrochloride) | 2 | mg |
| Zinc (as zinc sulphate monohydrate) | 10 | mg |
| Cyanocobalamine | 1 | μg |

wherein glycerol monostearate, lecithin, and gelatin are added in a total amount of 1806 mg.

Further conventional adjuvants such as stabilizers and/or stability additives and/or production additives are included, if desired, in addition to the indicated ingredients.

### EXAMPLE 10

Three capsule shells of the recommended daily dose of 712 mg in total are composed of glycerol, gelatine, red iron oxide. Further conventional adjuvants such as stabilizers and/or stability additives and/or production additives are included, if desired, in addition to the indicated ingredients.

### EXAMPLE 11

A recommended daily dose of 3 capsules contain:

| | | |
|---|---|---|
| Fish oil | 1425 | mg, |
| Omega-3 fatty acids (calculated as triglycerides) | 855 | mg |
| Borage seed oil | 88 | mg, |
| Gamma-linolenic acid | 15 | mg |
| (R,R,R)-alpha-tocopherol concentrate | 10 | mg |
| Ascorbic acid (as calcium ascorbate) | 60 | mg |
| Vitamin B6 (as pyridoxinehydrochloride) | 2 | mg |
| Zinc (as zinc sulphate monohydrate) | 10 | mg |
| Cyanocobalamine | 1 | μg |

wherein glycerol monostearate, lecithin, gelatine are added ad 1806 mg.

Further conventional adjuvants such as stabilizers and/or stability additives and/or production additives are included, if desired, in addition to the indicated ingredients.

### EXAMPLE 12

Three capsule shells of the recommended daily dose of 713.6 mg in total are composed of glycerol, gelatine, brilliant blue dye. Further conventional adjuvants such as stabilizers and/or stability additives and/or production additives are included, if desired, in addition to the indicated ingredients.

While specific embodiments of the present invention have been described in the foregoing, it will be appreciated by those skilled in the art that many equivalents, modifications, substitutions, and variations may be made thereto without departing from the spirit and scope of the invention as defined in the appended claims.

What is claimed is:

1. A composition comprising: at least an omega-3 fatty acid, at least an omega-6 fatty acid, and zinc, the composition being suitable for treatment or amelioration of a dry eye condition.

2. The composition of claim 1, wherein the composition further comprises at least one vitamin selected from the group consisting of vitamin E, vitamin C, vitamin B₆, vitamin B₁₂, and combination thereof.

3. The composition of claim 2, wherein said at least an omega-3-fatty acid comprises fish oil triglycerides, eicosapentaenoic acid, docosahexaenoic acid, or a combination thereof.

4. The composition of claim 3, wherein said at least an omega-6 fatty acid is gamma-linolenic acid.

5. The composition of claim 4, wherein a weight ratio of said at least an omega-3 fatty acid, calculated as triglycerides, and said gamma-linolenic acid is from about 3:1 to about 200:1.

6. The composition of claim 1, wherein said at least an omega-3-fatty acid comprises fish oil triglycerides, eicosapentaenoic acid, docosahexaenoic acid, or a combination thereof.

7. The composition of claim 6, wherein the omega-6 fatty acid is gamma-linolenic acid.

8. A composition, based on a single dose of the composition, comprising:
(a) at least an omega-3 fatty acid in an amount from about 10 to about 500 mg;
(b) gamma-linolenic acid in an amount from about 1 to about 10 mg;
(c) vitamin E in an amount from about 1 to about 5 mg;
(d) vitamin C in an amount from about 1 to about 50 mg;
(e) vitamin B₆ in an amount from about 0.1 to about 1 mg;
(f) vitamin B₁₂ in an amount from about 0.01 μg to about 1 μg; and
(g) zinc in an amount from about 0.1 to about 10 mg.

9. The composition of claim 8, wherein the amount of omega-3 fatty acid is from about 200 to about 300 mg; the amount of gamma-linolenic acid is from about 4 to about 6 mg; the amount of vitamin E is from about 2 to about 4.5 mg; the amount of vitamin C is from about 15 to about 30 mg; the amount of vitamin B₆ is from about 0.3 to about 0.8 mg; the amount of vitamin B₁₂ is from about 0.15 μg to about 0.7 μg; and the amount of zinc is from about 1.5 to about 5 mg.

10. The composition of claim 8, wherein the weight ratio of said at least an omega-3 fatty acid, calculated as triglycerides, and the gamma-linolenic acid is from about 3:1 to about 200:1.

11. The composition of claim 8, wherein the weight ratio of said at least an omega-3 fatty acid, calculated as triglycerides, and the gamma-linolenic acid is from about 20:1 to about 100:1.

12. The composition of claim 7, wherein the composition is in a form of a solid, liquid, or gel.

13. The composition of claim 8, wherein the composition has a total weight from about 200 to about 1500 mg, excluding any shell or coating.

14. The composition 13, wherein the composition further comprises an adjuvant selected from the group consisting of glycerol monostearate, lecithin, gelatin, and a combination thereof.

15. A method for treating or ameliorating a dry eye condition, comprising administering to a subject in need thereof a composition of claim 1.

16. The method of claim 15, wherein the composition further comprises at least one vitamin selected from the group consisting of vitamin E, vitamin C, vitamin B₆, vitamin B₁₂, and combination thereof.

17. The method of claim 16; wherein said at least an omega-3-fatty acid comprises fish oil triglycerides, eicosapentaenoic acid, docosahexaenoic acid, or a combination thereof; said at least an omega-6 fatty acid comprises gamma-linolenic acid; and a weight ratio of said at least an omega-3 fatty acid, calculated as triglycerides, and said gamma-linolenic acid is from about 3:1 to about 200:1.

18. A method for treating or ameliorating a dry eye condition, comprising administering to a subject in need thereof a composition that comprises, based on a single dose of the composition:
(a) at least an omega-3 fatty acid in an amount from about 10 to about 500 mg;
(b) gamma-linolenic acid in an amount from about 1 to about 10 mg;
(c) vitamin E in an amount from about 1 to about 5 mg;
(d) vitamin C in an amount from about 1 to about 50 mg;
(e) vitamin B₆ in an amount from about 0.1 to about 1 mg;
(f) vitamin B₁₂ in an amount from about 0.01 μg to about 1 μg; and
(g) zinc in an amount from about 0.1 to about 10 mg.

19. The method of claim 18; wherein the amount of omega-3 fatty acid is from about 200 to about 300 mg; the amount of gamma-linolenic acid is from about 4 to about 6 mg; the amount of vitamin E is from about 2 to about 4.5 mg; the amount of vitamin C is from about 15 to about 30 mg; the amount of vitamin B₆ is from about 0.3 to about 0.8 mg; the amount of vitamin B₁₂ is from about 0.15 μg to about 0.7 μg; and the amount of zinc is from about 1.5 to about 5 mg.

20. The method of claim 18; wherein said at least an omega-3-fatty acid comprises fish oil triglycerides, eicosapentaenoic acid, docosahexaenoic acid, or a combination thereof; said at least an omega-6 fatty acid comprises gamma-linolenic acid; and a weight ratio of said at least an omega-3 fatty acid, calculated as triglycerides, and said gamma-linolenic acid is from about 20:1 to about 100:1.
